SEQUENCE LISTING (1 sequence)

SEQ ID NO: 1 - protein, 12 aa, Artificial Sequence
  Synthetic sequence, partial peptide of collagen
  misc_feature (3)..(3): Xaa is 4-Hydroxyproline
  misc_feature (6)..(6): Xaa is 4-Hydroxyproline
GPXGPXGASGPQ

(19) United States
(12) Patent Application Publication
(10) Pub. No.: US 2021/0228675 A1
(43) Pub. Date: Jul. 29, 2021

(54) HYPERTROPHIC SCAR INHIBITING COMPOSITION

(71) Applicants: FUKUOKA UNIVERSITY, Fukuoka-shi, Fukuoka (JP); NITTA GELATIN INC., Osaka-shi, Osaka (JP)

(72) Inventors: Shiro JIMI, Fukuoka-shi, Fukuoka (JP); Masahiko KIMURA, Fukuoka-shi, Fukuoka (JP); Naoki INOUE, Yao-shi, Osaka (JP); Maiko SHIMIZU, Yao-shi, Osaka (JP); Seiko KOIZUMI, Yao-shi, Osaka (JP)

(73) Assignees: FUKUOKA UNIVERSITY, Fukuoka-shi, Fukuoka (JP); NITTA GELATIN INC., Osaka-shi, Osaka (JP)

(21) Appl. No.: 16/972,381

(22) PCT Filed: May 28, 2019
(86) PCT No.: PCT/JP2019/020997

§ 371 Date: Dec. 4, 2020

(30) Foreign Application Priority Data
Jun. 8, 2018 (JP) 2018-110374

(51) Int. Cl. A61K 38/05; A61K 9/00; A61K 38/39; A61P 17/02

(52) U.S. Cl. CPC A61K 38/05; A61P 17/02; A61K 38/39; A61K 9/0053

(57) ABSTRACT

A composition for inhibiting the formation of hypertrophic scar, comprising at least one of a polypeptide consisting of an amino acid sequence having a dipeptide sequence represented by Pro-Hyp or Hyp-Gly, a chemically-modified form thereof, or a pharmaceutically acceptable salt thereof.

### TECHNICAL FIELD

The present invention relates to a composition for inhibiting the formation of hypertrophic scar.

### BACKGROUND ART

The healing process of wounds (more specifically, acute wounds such as cuts, abrasions, and burns and chronic wounds such as decubitus ulcers) in the skin or the like is usually classified into 5 steps of the clotting and hemostasis phase, the inflammatory phase, the proliferative phase, the tissue reconstruction phase, and the maturation phase. In the proliferative phase, fibroblasts and capillaries infiltrate the wound site, and the proliferation of fibroblasts and the production of collagen fibers are promoted. As a result, granulation tissue is formed in the wound site in the proliferative phase.

The granulation tissue formed in the proliferative phase retracts after a while in the subsequent tissue reconstruction phase and the maturation phase and is replaced by healed tissue in the end. However, a hypertrophic scar (including keloid) is known to be formed, if an abnormality, such as the excessive formation of the granulation tissue in the proliferative phase, occurs in the wound healing process.

### CITATION LIST

### Non Patent Literature

NPL 1: Rei Ogawa et al., “Role of Mechanical Forces and Its Molecular Mechanisms in Wound Healing: —Mechanobiology and Mechanotherapy—”, Japanese Journal of Surgical Wound Care, 5 (3): 102-107, 2014

### SUMMARY OF INVENTION

### Technical Problem

In terms of the aesthetic appearance of the skin or quality of life (QOL), methods for inhibiting the formation of hypertrophic scar have been studied. However, the mechanism of formation of the granulation tissue described above has been not yet understood sufficiently. In conventional arts, only known methods for inhibiting the formation of hypertrophic scar are methods of physical treatments such as resting, fixation, and compression of the wound (for example, Japanese Journal of Surgical Wound Care, 5 (3): 102-107, 2014 (Non Patent Literature 1)). Therefore, the development of a composition for inhibiting the formation of the hypertrophic scar and the like is desired.

The present invention has been made in view of such circumstances and an object of the invention is to provide a composition for inhibiting the formation of hypertrophic scar.

### Solution to Problem

The present inventors have studied diligently to achieve the aforementioned object and have found, as a result, that polypeptides having particular sequences effectively inhibit the formation of hypertrophic scar, thereby completing the present invention. Accordingly, the present invention is as follows.

[1] A composition for inhibiting a formation of hypertrophic scar, comprising at least one of a polypeptide consisting of an amino acid sequence having a dipeptide sequence represented by Pro-Hyp or Hyp-Gly, a chemically-modified form thereof, or a pharmaceutically acceptable salt thereof.

[2] The composition for inhibiting the formation of hypertrophic scar according to [1], wherein the composition is used to inhibit the formation of hypertrophic scar at a site susceptible to mechanical stress.

[3] The composition for inhibiting the formation of hypertrophic scar according to [2], wherein the site susceptible to mechanical stress comprises at least one selected from the group consisting of the abdomen, chest, upper arms, face, and soft tissues.

[4] The composition for inhibiting the formation of hypertrophic scar according to any of [1] to [3], wherein the polypeptide comprises an oligopeptide consisting of an amino acid sequence of 2 to 20 amino acid residues.

[5] The composition for inhibiting formation of hypertrophic scar according to any of [1] to [4], wherein the polypeptide comprises a dipeptide consisting of an amino acid sequence represented by Pro-Hyp or Hyp-Gly.

[6] The composition for inhibiting the formation of hypertrophic scar according to any of [1] to [5], wherein the polypeptide is a polypeptide derived from natural collagen, a recombinant polypeptide, or a synthetic polypeptide.

[7] The composition for inhibiting the formation of hypertrophic scar according to any of [1] to [6], wherein the composition is an oral administration formulation, a supplement, a food, or a beverage.

[8] The composition for inhibiting formation of hypertrophic scar according to any of [1] to [6], wherein the composition is a transdermal administration formulation, a local administration formulation, an intravenous administration formulation, or a cosmetic preparation.

[9] A method for inhibiting the formation of hypertrophic scar, comprising administering an effective amount of at least one of a polypeptide consisting of an amino acid sequence having a dipeptide sequence represented by Pro-Hyp or Hyp-Gly, a chemically-modified form thereof, or a pharmaceutically acceptable salt thereof to a subject in need thereof.

[10] Use of at least one of a polypeptide consisting of an amino acid sequence having a dipeptide sequence represented by Pro-Hyp or Hyp-Gly, a chemically-modified form thereof, or a pharmaceutically acceptable salt thereof for producing a composition for inhibiting formation of hypertrophic scar.

[11] At least one of a polypeptide consisting of an amino acid sequence having a dipeptide sequence represented by Pro-Hyp or Hyp-Gly, a chemically-modified form thereof, or a pharmaceutically acceptable salt thereof, for inhibiting formation of hypertrophic scar.

### Advantageous Effects of Invention

According to the present invention, it is possible to provide a composition for inhibiting the formation of hypertrophic scar.

### BRIEF DESCRIPTION OF DRAWINGS

FIG. 1 illustrates photographs of abdominal wall incision wound model mice used in Examples.

FIG. 2 is a set of photographs illustrating the histological change at a wound site.

FIG. 3 is a graph illustrating the change over time of blood concentration of the Pro-Hyp dipeptide in a mouse after intraperitoneal administration or oral administration of the dipeptide.

FIG. 4 is a schematic diagram illustrating a schedule of administration to mice.

FIG. 5 is a set of photographs illustrating the histological change at a wound site.

FIG. 6 is a set of photographs illustrating the distribution of collagen in a granulation tissue.

FIG. 7 is a pair of photographs illustrating the abdomen after healing of a wound site in mice of an abdominal wall incision wound model.

FIG. 8 is a set of photographs illustrating the change over time of a wound site in a view from the abdominal wall side in mice of an abdominal wall incision wound model.

FIG. 9 is a set of photographs illustrating the distribution of collagen in granulation tissue.

### DESCRIPTION OF EMBODIMENTS

An embodiment (hereinafter, also referred to as “the present embodiment”) of the present invention will be described below, but the present invention is not limited thereto. Here, an expression in a form of “A to B” herein indicates the upper and lower limits of the range (that is, equal to or higher than A and equal to or lower than B) and, if no unit is indicated in association with A, but a unit is only indicated in association with B, then the unit of A is the same as the unit of B.

“Pro”, “Hyp”, and “Gly” respectively mean proline, 4-hydroxyproline, and glycine.

<<Composition for Inhibiting the Formation of Hypertrophic Scar>>

The composition for inhibiting formation of hypertrophic scar according to the present embodiment comprises at least one of a polypeptide consisting of an amino acid sequence having a dipeptide sequence represented by Pro-Hyp or Hyp-Gly (which may be hereinafter referred to simply as “polypeptide”), a chemically-modified form thereof, or a pharmaceutically acceptable salt thereof.

The polypeptide comprises, in the amino acid sequence thereof, a dipeptide sequence represented by Pro-Hyp or Hyp-Gly. Therefore, for example, after transdermal administration or oral administration, the aforementioned polypeptide is degraded in the body and a dipeptide consisting of an amino acid sequence represented by Pro-Hyp or Hyp-Gly is produced. And the present inventors consider that the produced dipeptide can reach the wound site through the blood to inhibit the formation of hypertrophic scar.

In the present embodiment, the “hypertrophic scar” means a raised scar formed after an injury by excessive production of fibrous tissue formed for repairing the wound (which may be hereinafter referred to as the “wound site”). Hypertrophic scars that expand to normal skin are particularly called “keloid”.

In the present embodiment, “inhibition of formation of hypertrophic scar” and “inhibit the formation of hypertrophic scar” mean that the formation of the hypertrophic scar is inhibited by inhibiting excessive proliferation of granulation tissue or excessive production of collagen fibers in the proliferative phase of the wound healing process, promoting the retraction of granulation tissue in the tissue reconstruction phase and the maturation phase of the wound healing process, or the like. Here, the “wound” means, for example, an acute wound such as a cut, an abrasion, or a burn, a chronic wound such as decubitus ulcer, or the like.

In the present embodiment, the “polypeptide” means a chain molecule formed by a peptide linkage of 2 or more amino acids. In the present embodiment, polypeptides consisting of amino acid sequences of 2 to 20 amino acid residues are called “oligopeptides”. Above all, oligopeptides formed by peptide linkage of 2 amino acids are called “dipeptides”. Oligopeptides formed by peptide linkage of 3 amino acids are called “tripeptides”.

The aforementioned polypeptide has, in the amino acid sequence thereof, a dipeptide sequence represented by Pro-Hyp or Hyp-Gly. The polypeptide may have 1 to 500 or 1 to 10 dipeptide sequences represented by Pro-Hyp or Hyp-Gly in the amino acid sequence thereof.

In the present embodiment, the aforementioned polypeptide preferably comprises an oligopeptide consisting of an amino acid sequence of 2 to 20 amino acid residues and more preferably comprises an oligopeptide consisting of an amino acid sequence of 2 to 15 amino acid residues. Having it an oligopeptide makes it possible to promote degradation in the body and absorption into the body, for example, when administered transdermally or orally, and to deliver a dipeptide consisting of an amino acid sequence represented by Pro-Hyp or Hyp-Gly efficiently to the wound site. Examples of the oligopeptide include an oligopeptide consisting of the following amino acid sequence.

(SEQ ID NO: 1)
Gly-Pro-Hyp-Gly-Pro-Hyp-Gly-Ala-Ser-Gly-Pro-Gln

On another side of the present embodiment, the polypeptide preferably comprises a dipeptide consisting of an amino acid sequence represented by Pro-Hyp or Hyp-Gly and more preferably comprises the dipeptide consisting of the amino acid sequence represented by Pro-Hyp. Having it a dipeptide makes it possible to deliver the dipeptide efficiently to the wound site upon local administration, as well as transdermal or oral administration or the like.

Moreover, methods for obtaining or producing the polypeptide are not particularly limited, but the polypeptide may be a polypeptide derived from natural collagen, a recombinant polypeptide, or a synthetic polypeptide.

Examples of “natural collagen” include collagen derived from a mammal such as cow and pigs, birds, and the like and collagen derived from fish such as shark, sea bream, and tilapia, and the like. These can be obtained from connective tissues such as parts of the aforementioned mammals and birds such as the bone, the skin, and the tendon; and parts of the aforementioned fish such as the bone, the skin, and the scale. Specifically, the aforementioned bone, skin, scale, or the like may be subjected to a conventionally known treatment such as a degreasing treatment, a decalcification treatment, or an extraction treatment.

Examples of the “polypeptide derived from natural collagen” include polypeptides obtained by hydrolyzing natural collagen by enzymatic treatment and the like. Examples of an enzyme to be used in the enzymatic treatment include collagenases, thiol proteases, serine proteases, acid proteases, alkaline proteases, metalloproteases, and the like. The enzymes may be used singly or a combination of two or more of the enzymes may be used. Examples of the thiol proteases include plant-derived chymopapain, papain, bromelain, ficin, animal-derived cathepsin, calcium-dependent proteases, and the like. Moreover, examples of the serine proteases include trypsin, cathepsin D, and the like. Examples of the acid proteases include pepsin, chymotrypsin, and the like. Considering that the obtained polypeptide is used in a medicine or food for specified health uses, the enzyme to be used is preferably an enzyme other than enzymes derived from pathogenic microorganisms (for example, an enzyme derived from a nonpathogenic microorganism). Examples of the nonpathogenic microorganism from which the enzyme is derived include Bacillus licheniforms, Bacillus subtillis, Aspergillus oryzae, Streptomyces, Bacillus amyloliquefaciens, and the like. The enzyme may be an enzyme derived from one of the nonpathogenic microorganisms or a combination of enzymes derived from two or more of the nonpathogenic microorganisms. Examples of Specific methods of the enzymatic treatment are those known conventionally.

Moreover, the peptide may be synthesized using a non-ribosomal peptide synthetase or the like.

The “recombinant polypeptide” means a polypeptide produced artificially using a gene recombination technique using Escherichia coli, yeast, cultured cells as a host. The method for producing the recombinant polypeptide may be a conventionally known method. Specific examples thereof include the following methods. First, a vector having a nucleotide sequence encoding a polypeptide of interest and a vector having a nucleotide sequence encoding an enzyme that hydroxylates an amino acid (for example, L-proline cis-4-hydroxylase) are introduced into host Escherichia coli to perform transformation. By culturing the transformed Escherichia coli in a predetermined medium, the polypeptide of interest is synthesized by the Escherichia coli.

Moreover, the following methods are possible as well. First, Escherichia coli having a peptide bond-forming enzyme is produced using a gene recombination technique and isolated after the synthesis of the enzyme by the Escherichia coli. By having the isolated enzyme reacted with the amino acid, a polypeptide having an intended amino acid sequence is synthesized. In the hydroxylation of the amino acid, a method using L-proline cis-4-hydroxylase, which is a conventional art, may be adopted.

The “synthesized polypeptide” means a polypeptide produced by coupling amino acids that are raw materials one by one. Examples of the method for synthesis from amino acids include methods of solid-phase synthesis and method of fluid-phase synthesis. Examples of the methods of solid-phase synthesis include the Fmoc method, the Boc method, and the like. The polypeptide according to the present embodiment may be synthesized by any of the methods.

For example, the polypeptide may be synthesized by a known method of solid-phase synthesis in which proline is immobilized onto carrier polystyrene and the fluorenyl-methoxy-carbonyl group (Fmoc group) or the tert-Butyl Oxy Carbonyl group (Boc group) is used for protection of the amino group. More specifically, beads of a polystyrene polymer gel with a diameter of 0.1 mm or so whose surface is modified with amino groups are used as a solid-phase and hydroxyproline is coupled (peptide-linked) to proline whose amino group is protected with an Fmoc group by dehydration using diisopropylcarbodiimide (DIC) as a condensing agent. Subsequently, the solid-phase is washed well with a solvent and the remaining hydroxyproline is removed. The dipeptide containing the sequence Pro-Hyp can be then synthesized by removing the proline protecting group bound to the solid-phase (deprotection). Subsequently, the tripeptide containing the sequence of Pro-Hyp-Gly can be obtained by coupling (peptide-linking) glycine to the amino group in the hydroxyproline residue of this dipeptide in a similar method. In this way, the polypeptide of interest can be synthesized by coupling amino acids sequentially.

In the present embodiment, a “chemically-modified form” of a polypeptide means a polypeptide in which an amino group(s), a carboxyl group(s), or a hydroxy group(s) in amino acid residues composing the polypeptide is chemically modified. A polypeptide subjected to chemical modification may change the solubility in water, the isoelectric point, or the like. Specific examples for the hydroxy group in the hydroxyproline residue include chemical modifications by O-acetylation and the like. Examples for the α-carboxyl group in the glycine residue include chemical modifications by esterification, amidation, and the like. Examples for the α-amino group in the proline residue include chemical modifications by polypeptidylation, succinylation, maleylation, acetylation, deaminaiton, benzoylation, alkylsulfonylation, allylsulfonylation, dinitrophenylation, trinitrophenylation, carbamylation, phenylcarbamylation, thiolation, and the like. Moreover, a particular peptide can be made basic by performing ethylenediamination, spermination, or the like.

Specific means and treatment conditions for the chemical modification of the polypeptide that are applied are of usual chemical modification techniques for polypeptides. For the chemical modification of the hydroxy group in the hydroxyproline residue, O-acetylation, for example, can be performed by having acetic anhydride reacting with the group in an aqueous solvent or a nonaqueous solvent, or the like. For the chemical modification of the α-carboxyl group in the glycine residue, esterification, for example, can be performed by suspending the polypeptide to methanol and then bubbling the suspension with a dried hydrogen chloride gas, or the like. For the chemical modification of the α-carboxyl group in the glycine residue, amidation can be performed by having carbodiimide reacting with the group.

In the present embodiment, the “pharmaceutically acceptable salt” of the polypeptide means a salt that is pharmaceutically acceptable and has a desired pharmacological activity (inhibition of formation of the hypertrophic scar) of the original polypeptide. Examples of the pharmaceutically acceptable salt include inorganic acid salts such as hydrochloride, sulfate, phosphate, and hydrobromide; organic acid salts such as acetate, methanesulfonate, benzenesulfonate, p-toluenesulfonate, succinate, oxalate, fumarate, and maleate; inorganic base salts such as sodium salts, potassium salts, and calcium salts; organic base salts such as triethylammonium salts, and the like. A particular peptide can be turned into a pharmaceutically acceptable salt according to a routine method.

The composition for inhibiting the formation of hypertrophic scar in the present embodiment comprises at least one of the aforementioned polypeptides, a chemically-modified form thereof, or a pharmaceutically acceptable salt thereof. More specifically, the composition for inhibiting the formation of the hypertrophic scar may comprise the aforementioned polypeptide alone, a chemically-modified form thereof, or a pharmaceutically acceptable salt thereof and another component (for example, a filler, a binder, a solvent, or the like described below) that composes the composition. Moreover, the composition for inhibiting the formation of the hypertrophic scar may comprise two or more of the aforementioned polypeptide, a chemically-modified form thereof, or a pharmaceutically acceptable salt thereof. When the composition for inhibiting formation of hypertrophic scar comprises two or more of the aforementioned polypeptide, a chemically-modified form thereof, or a pharmaceutically acceptable salt thereof, the composition for inhibiting formation of hypertrophic scar may further comprise another component described above.

For example, the composition for inhibiting the formation of the hypertrophic scar may comprise a first polypeptide and a chemically-modified form of a second polypeptide. The composition for inhibiting the formation of the hypertrophic scar may comprise a first polypeptide, a chemically-modified form of a second polypeptide, and a pharmaceutically acceptable salt of a third polypeptide. Moreover, the composition for inhibiting formation of hypertrophic scar may be a polypeptide mixture containing two or more polypeptides. The polypeptide mixture described above obtained by hydrolyzing collagen may be called a “collagen hydrolysate”.

The polypeptide mixture may be a commercially available product. Examples of the commercially available product include, but are not limited to, IXOS HDL-50SP (trade name), SCP-5200 (trade name), Collapep JB (trade name) and IXOS HDL-12SP (trade name), TYPE-S (trade name), Collapep PU (trade name), and the like manufactured by Nitta Gelatin Inc.

The weight average molecular weight of the polypeptide mixture is preferably 130 to 7000 and more preferably 150 to 6500. The weight average molecular weight can be determined, for example, by gel filtration chromatography.

Specifically, the weight average molecular weight can be determined by performing measurement by gel filtration chromatography under the following conditions.

Mobile phase: 45% acetonitrile (55% water) containing 0.1% trifluoroacetic acid Stationary phase: a TSK-Gel-2000SWXL column (manufactured by Tosoh Corporation)
Flow rate: 1.0 ml/min,
Column temperature: 40° C.,
Analysis time: 15 minutes,
Injection volume: 10 μl,
Detection wavelength: 214 nm

The composition for inhibiting the formation of the hypertrophic scar is preferably used to inhibit the formation of hypertrophic scar at a site susceptible to mechanical stress. Here, the “mechanical stress” means physical force that occurs in the skin, a soft tissue, or the like in a natural state. Examples of the physical force include pressure, tension, shear stress, hydrostatic pressure, osmotic pressure, and the like. Here, the “soft tissue” means supporting tissue other than the skeleton and examples thereof include tendons, ligaments, muscular fasciae, adipose tissues, blood vessels, muscles (for example, striated muscles, smooth muscles).

The site susceptible to mechanical stress is preferably a site in the skin or soft tissue where the expansion and contraction occur frequently.

In the present embodiment, the site susceptible to mechanical stress preferably comprises at least one selected from the group consisting of the abdomen, chest, upper arms, face, and soft tissues.

Here, the process of wound healing at a site susceptible to mechanical stress is described in an example of the abdominal wound. Since the abdomen is under mechanical stress such as tension, the formation of a wound in the abdomen results in the expansion of the wound site by tension. What is important for curing the wound site that has expanded under mechanical stress such as tension is to (1) fill the wound site with granulation tissue and (2) contract the wound site that has expanded under mechanical stress against mechanical stress. In such a situation, usually, granulation tissue first proliferates and covers the wound site. Then, the contraction of the wound site and accompanying retraction of granulation tissue occurs and finally, the wound heals. In this process, if the granulation tissue proliferates excessively, then the retraction of the granulation tissue does not occur sufficiently in the tissue reconstruction phase and the maturation phase, consequently, a hypertrophic scar is formed.

Only methods conventionally known for inhibiting the formation of hypertrophic scar have been those involving physical treatments such as the rest, fixation, compression, and the like of the wound site. However, upon the use of the composition for inhibiting the formation of hypertrophic scar according to the present embodiment, the aforementioned dipeptide, which is an active ingredient, first promotes the contraction of the wound site that has expanded under mechanical stress. As a result, a less amount of granulation tissue that covers the wound site is required and this results in the inhibition of the proliferation of the granulation tissue. Finally, the retraction of granulation tissue occurs sufficiently in the tissue reconstruction phase and the maturation phase, and the formation of the hypertrophic scar is inhibited. This is a view of the present inventors.

The composition for inhibiting the formation of the hypertrophic scar may be an oral administration formulation, a supplement, a food, or a beverage.

Examples of the dosage form of the oral administration formulation include tablets, granules, capsules, powders, powdered medicines, liquids, and solutions.

Examples of pharmaceutical carriers for oral administration formulations include those used conventionally such as fillers (crystalline cellulose, lactose, sugar, cornstarch, potassium phosphate, Sorbit, glycine, and the like), binders (syrup, gum arabic, Sorbit, tragacanth, polyvinylpyrrolidone, and the like), lubricants (magnesium stearate, talc, polyethyleneglycol, silica, and the like), disintegrants (potato starch, and the like), and moistening agents (sodium lauryl sulfate, and the like).

Examples of the supplement include tablets, granules, capsules, powders, powdered medicines, liquids, and solutions.

Examples of the food include confectioneries such as candies, gums, tablets, and snack foods, ices such as ice creams and sherbets, cooked rice such as rice cakes and instant cooked rice, noodles such as udon, ramen, and pasta, soups such as instant soups and potage. Moreover, the food may be a food for specified health uses.

Examples of the beverage include fruit juices, tea-based beverages, coffee beverages, soft drinks, milk beverages, lactic fermenting beverages, carbonated beverages, nutritional beverages, and the like.

When the composition for inhibiting the formation of hypertrophic scar in the present embodiment is an oral administration formulation, a supplement, a food, or a beverage, the content ratio of the polypeptide relative to the composition for inhibiting the formation of hypertrophic scar in total is preferably 0.0001 to 100% by mass and more preferably 0.001 to 80% by mass. When the composition is used for a supplement for ingestion, the content ratio of the polypeptide is preferably 0.001 to 80% by mass for tablets, preferably 0.001 to 80% by mass for granules, preferably 0.1 to 30% by mass for capsules, preferably 0.001 to 100% by mass for powders and powdered medicines, and preferably 0.1 to 30% by mass for the liquids and solutions. Moreover, when the composition is used for a food, the content ratio of the polypeptide is preferably 0.001 to 30% by mass for candies and gums, preferably 0.001 to 80% by mass for tablets, preferably 0.0001 to 30% by mass for snack foods, ices, and cooked rice, preferably 0.0001 to 20% by mass for noodles, and preferably 0.0001 to 50% by mass for soups. Furthermore, when the composition is used for a beverage, the content ratio of the polypeptide is preferably 0.0001 to 50% by mass for fruit juices, tea-based beverages, coffee beverages, soft drinks, milk beverages, lactic fermenting beverages and nutritious supplement beverages.

Moreover, the composition for inhibiting the formation of the hypertrophic scar may be a transdermal administration formulation, a local administration formulation, an intravenous administration formulation, or a cosmetic preparation.

Examples of the dosage form of the transdermal administration formulation or the local administration formulation include creams, gels, ointments, liquids and solutions, liniments, emulsions, air sprays, drops, patches, dressings, injections, and the like. The transdermal administration formulation may additionally contain an appropriate filler as needed. The filler may be of any kind, as long as it is usually used in pharmaceutical preparations, medical devices, quasi drugs, and the like. Examples of the filler include polyvinyl alcohol, glycerol, chitosan, carboxymethylcellulose, hyaluronic acid, polypropyleneglycol, and the like.

Examples of the dosage form of the intravenous administration formulation include injections, drops, and the like. The injections and drops include solutions, suspensions, emulsions, and solid preparations to be solved or suspended into a solvent to be used when needed. The intravenous administration formulation is obtained by dissolving, suspending, or emulsifying the polypeptide in a solvent and then used. Examples of the solvent include distilled water for injection, physiological saline, vegetable oils, propylene glycol, polyethyleneglycol, alcohols such as ethanol, and the like and combinations thereof. Furthermore, the intravenous administration formulation may comprise a stabilizer, a solubilizing agent (glutamic acid, aspartic acid, polysorbate 80 (R), or the like), a suspending agent, an emulsifier, a soothing agent, a buffer, a preservative, or the like.

Examples of cosmetic preparation include lotions, emulsions, liquid cosmetics, packs, foundations, and the like.

When the composition for inhibiting the formation of hypertrophic scar in the present embodiment is a transdermal administration formulation, a local administration formulation, an intravenous administration formulation, or a cosmetic preparation, the content ratio of the polypeptide relative to the composition for inhibiting the formation of hypertrophic scar in total is preferably 0.000001 to 100% by mass and more preferably 0.001 to 20% by mass. When the composition is administered transdermally or locally, the content ratio of the polypeptide is preferably 0.0001 to 10% by mass for creams, gels, and ointments, preferably 0.0001 to 20% by mass for liquids and solutions, emulsions, propellants, and patches, preferably 0.01 to 5% by mass for dressings, and preferably 0.0001 to 0.5% by mass for injections. Moreover, when the composition is administered intravenously, the content ratio of the polypeptide is preferably 0.0001 to 0.5% by mass for injections and drops. When the composition is used for a cosmetic preparation, the content ratio of the polypeptide is preferably 0.0001 to 5% by mass for lotions and emulsions, preferably 0.0001 to 100% by mass for liquid cosmetics and packs, and preferably 0.000001 to 1% by mass for makeup products such as foundations.

<<Method for Inhibiting Formation of Hypertrophic Scar>>

The method for inhibiting the formation of hypertrophic scar according to the present embodiment comprises administering an effective amount of at least one of a polypeptide consisting of an amino acid sequence having a dipeptide sequence represented by Pro-Hyp or Hyp-Gly, a chemically-modified form thereof, or a pharmaceutically acceptable salt thereof to a subject in need thereof.

Here, the “effective amount” means a total amount of the aforementioned polypeptide, a chemically-modified form thereof, and a pharmaceutically acceptable salt thereof required for inhibiting the formation of hypertrophic scar.

Examples of the subject include mammals such as mice, rats, rabbits, cats, dogs, cows, horses, monkeys, and humans. The subject is preferably a human.

Examples of the administration route of the aforementioned polypeptide include oral administration, transdermal administration, local administration, intravenous administration, intraperitoneal administration, and the like.

The dose of the aforementioned polypeptide varies according to the age, the sex, the body weight, the difference of sensitivity of the subject, the mode of administration, the administration interval, the kind of active ingredient, the kind of formulation, and the like. When the aforementioned polypeptide is administered orally, for example, the dose thereof is preferably 0.01 to 50000 mg/kg and more preferably 1 to 500 mg/kg per day for an adult.

Moreover, when the aforementioned polypeptide is a dipeptide consisting of an amino acid sequence represented by Pro-Hyp or Hyp-Gly and another dose unit is used, the dose by oral administration may be, for example, 0.0001 to 70000 μmol/kg or 0.01 to 700 μmol/kg per day for an adult.

When the aforementioned polypeptide is administered transdermally, locally, intravenously, or intraperitoneally, the dose thereof is, for example, preferably 0.01 to 150 mg/kg and more preferably 1 to 15 mg/kg per day for an adult.

Moreover, when the aforementioned polypeptide is a dipeptide consisting of an amino acid sequence represented by Pro-Hyp or Hyp-Gly and another dose unit is used, the dose by transdermal administration, local administration, intravenous administration, or intraperitoneal administration may be, for example, 0.01 to 6000 μmol/kg or 1 to 60 μmol/kg per day for an adult.

The frequency of administration of the aforementioned polypeptide is not particularly limited, but it may be, for example, once a week, once in 3 days, or once a day.

The method for inhibiting the formation of hypertrophic scar according to the present embodiment may comprise, in conjunction with administering an effective amount of the aforementioned polypeptide or the like to a subject, providing another treatment. Examples of the other treatment include suture of the wound site, compression of the wound site, wet therapies, and the like.

### Other Aspects

An example of other aspects of the present embodiment is the use of at least one of polypeptides consisting of an amino acid sequence having a dipeptide sequence represented by Pro-Hyp or Hyp-Gly, a chemically-modified form thereof, or a pharmaceutically acceptable salt thereof for producing a composition for inhibiting the formation of hypertrophic scar.

Another example of other aspects of the present embodiment is at least one of a polypeptide consisting of an amino acid sequence having a dipeptide sequence represented by Pro-Hyp or Hyp-Gly, a chemically-modified form thereof, or a pharmaceutically acceptable salt thereof, for inhibiting the formation of hypertrophic scar.

### EXAMPLES

Hereinafter, the present invention will be described in detail with reference to Examples, but the present invention is not limited by these Examples.

<<Experiments Using Abdominal Wall Midline Incision Model (Model-1) Mice>>

<Generation of Model-1 Mice>

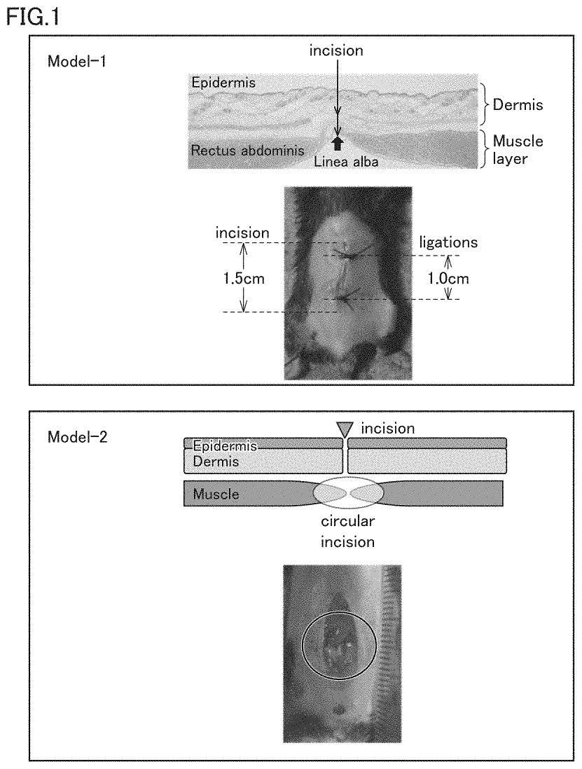

Female C57 BL/6N mice at 8 to 10 weeks of age were used. 200 μL per mouse (0.065 mg/g weight) of the anesthetic Somnopentyl (manufactured by Shering-Plough Corporation) was administered to the peritoneal cavity. Then, the abdominal skin of the mice was cut open 1.5 cm along the midline using scissors for operations (formation of wound site). Here, the abdomen is a site where expansion and contraction of the skin occur frequently and that is susceptible to mechanical stress. After the incision, the positions 0.25 cm apart from the upper end and 0.25 cm apart from the bottom end of the incised skin (which may be hereinafter referred to as the “wound site”) were sewed up to remove the tension of the skin (the upper panel of FIG. 1). Subsequently, the skin was wrapped with Tegaderm (trade name, trademark) manufactured by 3M Company and an underwear-shaped silicon protector was further put on to prevent the self-harm of the wound site. The following experiments were conducted using the Model-1 mice generated in this way.

<Preliminary Experiments>

### (1) Observation of Histological Change of Wound Site

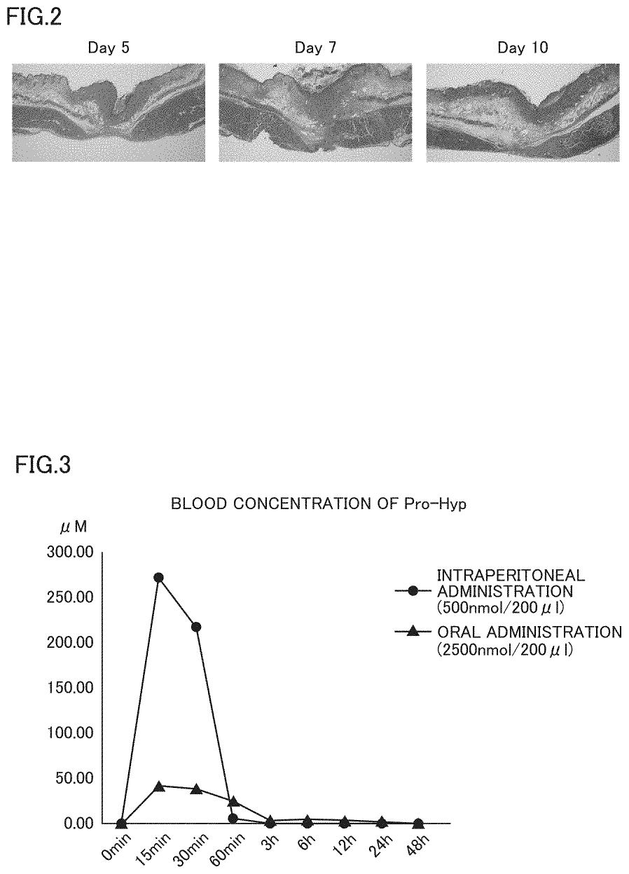

Using Model-1 mice, the histological change of the wound site was observed without administering anything after the formation of the wound site. Specifically, the Model-1 mice (N=4) were killed by blood removal under anesthesia respectively on Day 3, 5, 7, and 10 after the formation of the wound site. The abdominal wall tissue containing the wound site was extirpated, extended, and then fixed by immersing it in a 5% formaldehyde solution overnight. The tissue after the fixation was embedded in paraffin according to a routine method and tissue sections were produced with a microtome. Subsequently, the produced tissue sections were stained by Masson trichrome stain. This staining colors differentially the cytoplasm with red and collagen fibers with blue. The stained tissue sections were observed under a microscope. On Day 3 after the formation of the wound site, the binding of the tissues in the wound site was incomplete and the site was unsuitable for the observation (not shown). However, on Day 5 after the formation of the wound site or later, the conjunction of the epidermis and the coalescence of the tissue in the wound site by granulation tissue had been progressing (FIG. 2). Since the complete healing of the wound site is after approximately 2 weeks or later, it was judged to be appropriate to observe the histological change (the change of granulation tissue) of the wound site on Day 5, 7, and 10 after the formation of the wound site.

(2) Transfer of Pro-Hyp in Blood after Administration

To C57 BL/6N mice (8 to 10 weeks of age, female) (N=4, per group) in which no wound site is formed, a physiological saline solution (500 nmol/200 μL) of the dipeptide consisting of the amino acid sequence of Pro-Hyp (which may be hereinafter referred to as simply “Pro-Hyp”) is administered intraperitoneally by injection (single administration) or a physiological saline solution (2500 nmol/200 μL) of Pro-Hyp was orally administered using a probe (single administration). After administration, blood was collected over time and Pro-Hyp occurred in blood was measured by LC-MS/MS method. The measurement conditions of LC-MS/MS method were as follows.

### Measurement Conditions of LC-MS/MS Method

Collected blood was 2 times diluted in physiological saline and then centrifugation was performed under conditions at 20° C., 500×g, for 15 minutes. To the supernatant recovered after the centrifugation, 3 volumes of 100% ethanol were added. The supernatant to which 100% ethanol had been added was centrifuged at 20° C., 13000 rpm, for 15 minutes to perform deproteinization. Subsequently, the deproteinized sample was 10 times diluted in 50 mM ammonium bicarbonate and analyzed by LC-MS/MS. For the LC conditions, the analysis was conducted using CAPCELL PAK C1 UG120 10 mm×I.D. 2.0 mm (OSAKA SODA CO., LTD.) as a guard column and the column Hypersil Gold PFP 150 mm×I.D. 2.1 mm, 5 μm (manufactured by Thermo Fisher Scientific, Inc.). The analysis conditions were Mobile phase A MeOH, Mobile phase B a 2% formic acid aqueous solution containing 2 mM ammonium acetate, injection volume 5 μL, and gradient of Table 1 below.

TABLE 1

| Time | Flow Rate | MOBILE PHASE B | MOBILE PHASE A |
|---|---|---|---|
| (min) | (μL/min) | (% BY VOLUME) | (% BY VOLUME) |
| Init. | 200 | 98 | 2 |
| 5 | 200 | 98 | 2 |
| 7.1 | 400 | 5 | 95 |
| 9 | 400 | 5 | 95 |
| 9.1 | 200 | 98 | 2 |
| 17 | 200 | 98 | 2 |

The MS/MS was conducted using a tandem mass spectrometer (TSQ Vantage (manufactured by Thermo Fisher Scientific, Inc.)) under conditions of Method of ionization: Positive ESI, Spray voltage: 3000 V, Vaporizer: 300° C., Sheath gas: 30, Aux gas: 15, Capillary temperature: 250° C., MRM conditions: Parent Mass 229.1, Product Mass 70, Collision energy: 29 ev, S-Lens: 68.

The measurement results are shown in FIG. 3. From the results of FIG. 3, it was revealed that both in the intraperitoneal administration group and the oral administration group, the blood concentration of Pro-Hyp reached a peak 15 minutes after the administration, but decreased 1 hour after the administration, and decreased to a baseline level 3 hours after the administration. Moreover, in the intraperitoneal administration, the Pro-Hyp concentration reached very high since it transferred in blood without gastrointestinal absorption. Meanwhile, it was shown that in the oral administration, transfer in the blood was surely exhibited, although transfer levels in blood were less than those in the intraperitoneal administration. From this result, it was supposed that the dipeptide transfers to a wound site via circulating blood not only by intraperitoneal administration, but also by oral administration. The C57 BL/6N mice in which no wound site was formed were used in this experiment, it is considered that a similar result is also obtained with Model-1 mice and Model-2 mice generated from the same line of mice.

<Inhibition Test of Hypertrophic Scar Formation>

### (1) Follow-Up of Granulation Tissue in the Wound Site

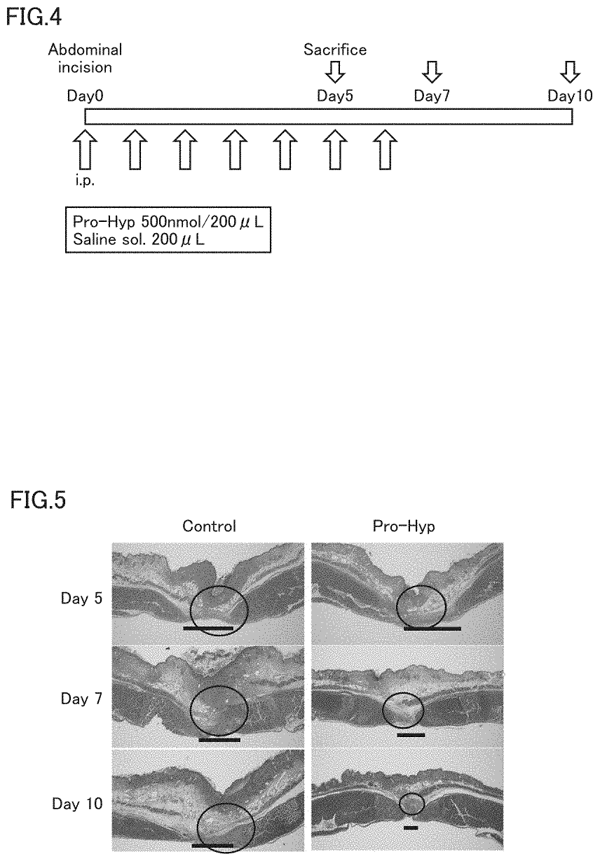

To the peritoneal cavity in Model-1 mice (N=5, per group) in which a wound site had been formed, an aqueous physiological saline solution of Pro-Hyp (500 nmol/200 μL) or a physiological saline containing no Pro-Hyp was administered. 200 μL each of either solution per animal was administered once a day for 7 days, including the day of operation (the day when a wound site was formed, Day 0) (FIG. 4). Mice were killed by blood removal under anesthesia respectively 5, 7, and 10 days (Day 5, 7, 10) after the formation of the wound site. The abdominal wall tissue containing the wound site was extirpated and extended. Then it was fixed by immersing it in a 5% formaldehyde solution overnight. The tissue after the fixation was embedded in paraffin according to a routine method and tissue sections were produced with a microtome. Subsequently, the produced tissue sections were stained by Masson trichrome stain. This staining colors differentially the cytoplasm with red and collagen fibers with blue. The stained tissue sections were observed under a microscope.

The results are shown in FIG. 5. By Day 5 after the formation of the wound site, most of the epidermal healing had been completed, but the healing on the peritoneal cavity side was still progressing. Generally, the granulation tissue in the wound site is larger (the part indicated with an oval in FIG. 5) in the control group (physiological saline administration group, Comparative Example) and the distance (the length of a bold line in FIG. 5) between the stumped recti abdominis, which is an indicator of diastasis of the wound site, is wider. Meanwhile, in the Pro-Hyp administration group (Example), the granulation tissue is smaller and the distance between the stumped recti abdominis is shorter. This result suggested that the proliferation of granulation tissue in the wound site is inhibited by the administration of Pro-Hyp.

### (2) Measurement of Distance Between the Stumped Recti Abdominis

From Masson trichrome stain images, the separation between the recti abdominis associated with healing from pathological observations was measured as the distance between the recti abdominis using an image analysis software (trade name: BZ-analyzer: manufactured by Keyence Corporation). The results are shown in Table 2. The distances between the stumped recti abdominis in Table 2 indicate the means determined with 5 mice per group. The distances (for example, FIG. 5) between the stumped recti abdominis did not vary largely through the experiment period in the control group. Meanwhile, the distances between the stumped recti abdominis in the Pro-Hyp administration group decreased in a time-dependent manner and significant differences from the control group were found both on Day 7 and Day 10 after the formation of the wound site.

TABLE 2

| | | | | TEST OF |
|---|---|---|---|---|
| | | | | SIGNIFICANT |
| | | | | DIFFERENCE |
| DISTANCE BETWEEN | | | STAN- | (CONTROL |
| THE STUMPED RECTI | | MEAN | DARD | GROUP VS. |
| ABDOMINIS | | (μm) | ERROR | Pro-Hyp GROUP) |
| Day | CONTROL GROUP | 695.5 | 127.8 | |
| 5 | Pro-Hyp GROUP | 532.8 | 159.0 | |
| Day | CONTROL GROUP | 812.3 | 135.3 | ** |
| 7 | Pro-Hyp GROUP | 358.3 | 73.0 | |
| Day | CONTROL GROUP | 707.5 | 205.3 | * |
| 10 | Pro-Hyp GROUP | 233.5 | 71.1 | |

* p < 0.05,
** p < 0.01 (student's t-test)

### (3) Measurement of the Area of Granulation Tissue

From the Masson trichrome stain images described above, the area of granulation tissue formed in the healing process from pathological observations was measured using an image analysis software (trade name: BZ-analyzer: manufactured by Keyence Corporation). The results are shown in Table 3. The areas of granulation tissue in Table 3 indicate the means determined from 5 mice per group. While the area of granulation tissue in the control group reached a peak on Day 7 after the formation of the wound site and then decreased, a decrease in a time-dependent manner was found in the Pro-Hyp group. Significant differences from the control group were found on Day 7 and Day 10 after the formation of the wound site in the Pro-Hyp group.

TABLE 3

| | | | | TEST OF |
|---|---|---|---|---|
| | | | | SIGNIFICANT |
| | | | | DIFFERENCE |
| AREA OF | | | STAN- | (CONTROL |
| GRANULATION | | MEAN | DARD | GROUP VS. |
| TISSUE | | (μm²) | ERROR | Pro-Hyp GROUP) |
| Day | CONTROL GROUP | 760020.2 | 170314.8 | |
| 5 | Pro-Hyp GROUP | 518871.0 | 84469.2 | |
| Day | CONTROL GROUP | 1290757.0 | 226996.1 | ** |
| 7 | Pro-Hyp GROUP | 486177.2 | 124228.0 | |
| Day | CONTROL GROUP | 888388.4 | 171070.7 | * |
| 10 | Pro-Hyp GROUP | 312793.2 | 91051.8 | |

* p < 0.05,
** p < 0.01 (student's t-test)

### (4) Distribution of Collagen in Granulation Tissue

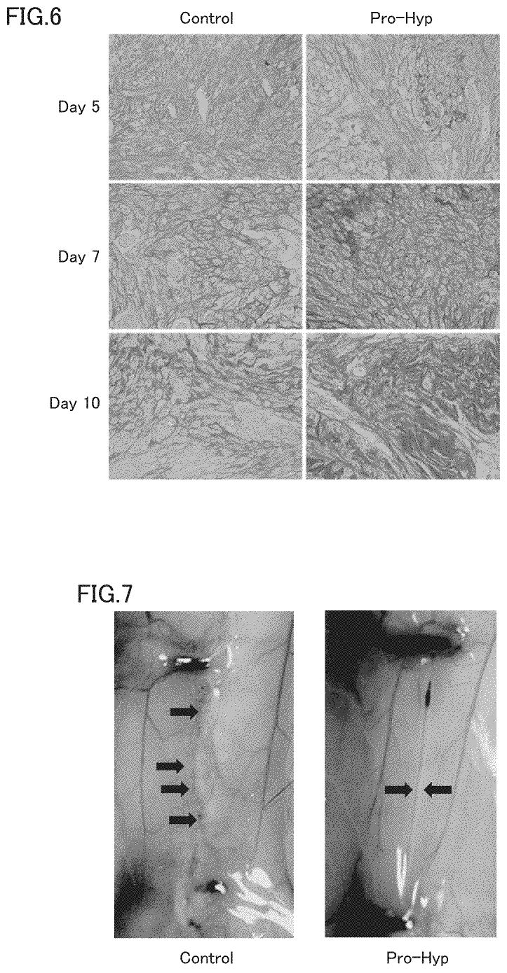

Using tissue sections prepared according to the method described in (1) above, staining with Picro-Sirius Red was conducted. This allows the staining of collagen fibers in granulation tissue in red. After the staining, pictures of granulation tissue in the tissue sections were taken. The results are shown in FIG. 6.

The collagen in early granulation tissue on Day 5 after the formation of the wound site is thin and distributes like reticular fibers. Subsequently, the staining grew stronger over time and thick collagen bundles had been formed by Day 10 after the formation of the wound site. This result suggested that collagen fibers that serve as the scaffolding of cells in granulation tissue in the process of the wound healing were first weak and thin collagen (type III collagen) and then changed into thick mature collagen (type I collagen).

### (5) Area of Collagen in Granulation Tissue

The Picro-Sirius Red staining images were binarized using an image processing software (trade name: Image J, manufactured by US National Institutes of Health). Fibrous collagen densely stained over the color threshold (≥150) was determined to be mature collagen (type I collagen) and the area of its distribution was measured to calculate the area of distribution of mature collagen per unit area in granulation tissue. The results are shown in Table 4. The distribution densities of collagen in Table 4 indicate the means determined from 5 mice per group. The collagen density in granulation tissue in the control group did not vary largely during the experiment period. Meanwhile, an increase of the distribution density of collagen in a time-dependent manner was found in the Pro-Hyp group and the maturation of collagen in granulation tissue was significant in the Pro-Hyp group.

TABLE 4

| | | | | TEST OF |
|---|---|---|---|---|
| | | | | SIGNIFICANT |
| | | | | DIFFERENCE |
| DISTRIBUTION | | | STAN- | (CONTROL |
| DENSITY | | MEAN | DARD | GROUP VS. |
| OF COLLAGEN | | (%) | ERROR | Pro-Hyp GROUP) |
| Day | CONTROL GROUP | 6.8 | 1.082 | |
| 5 | Pro-Hyp GROUP | 3.2 | 0.835 | |
| Day | CONTROL GROUP | 8.2 | 0.535 | |
| 7 | Pro-Hyp GROUP | 21.2 | 4.515 | |
| Day | CONTROL GROUP | 8.9 | 3.562 | * |
| 10 | Pro-Hyp GROUP | 26.5 | 2.726 | |

* p < 0.05 (student's t-test)

(6) Observation of Wound Site Viewing from the Abdominal Wall Side after Pro-Hyp Administration

The observation image of the wound site by viewing after the Pro-Hyp administration was compared with that of the control group (FIG. 7). Mice on Day 10 after the formation of the wound site were observed. In the tissue repair part after incision in the control group, whitish meandering granulation remained thickly (the part indicated by the arrows in the photograph in the left panel in FIG. 7). Meanwhile, the tissue repair part in the Pro-Hyp administration group appeared in a smooth straight line and exhibited healing with less granulation tissue (the part indicated by the arrows in the photograph in the right panel in FIG. 7). Accordingly, it was indicated that the formation of the hypertrophic scar can be inhibited by administering the polypeptide (dipeptide) consisting of the amino acid sequence having the dipeptide sequence represented by Pro-Hyp.

<<Experiment Using Abdominal Wall Circular Incision Model (Model-2) Mice>>

<Generation of Model-2 Mice>

In the abdominal wall midline incision model (Model-1), the formation of granulation tissue is seen in the skin tissue and the rectus abdominis tissue in the wound site, and sometimes it is difficult to distinguish which tissue the granulation tissue is derived from. In this Example, aiming to examine granulation tissue formed in the peritoneum layer under tension, a novel model (Model-2) of granulation tissue formation that occurs between the peritoneum layers, which are under the minimal effect of the healing response from the skin layer and under direct influence of the tension of muscles, was generated in the following procedures. Female C57 BL/6N mice at 8 to 10 weeks of age were used. Anesthesia was introduced by administering 200 μL per animal (0.065 mg/g weight) of the anesthetic Somnopentyl (manufactured by Shering-Plough Corporation) to the peritoneal cavity. After the introduction of anesthesia, abdominal skin was cut open 1.5 cm along the midline using scissors for operations. Here, the abdomen is a site where expansion and contraction of the skin occur frequently and that is susceptible to mechanical stress. Subsequently, the center part of the abdominal wall was picked up with round tip tweezers with an outside diameter of 3 mm, and the tissue in the central part of the abdominal wall was completely removed surgically in a circle with curved tip micro-operation scissors (Bottom panel in FIG. 1). After the incision, the positions 0.25 cm apart from the upper end and 0.25 cm apart from the bottom end of the incised skin (wound site) were sewed up to remove the tension of the skin. Subsequently, the skin was wrapped with Tegaderm and an underwear-shaped silicon protector was further put on to prevent the self-harm of the wound site. The following experiments were conducted using the Model-2 mice generated in this way.

<Evaluation Experiment of Contraction of Wound Site>

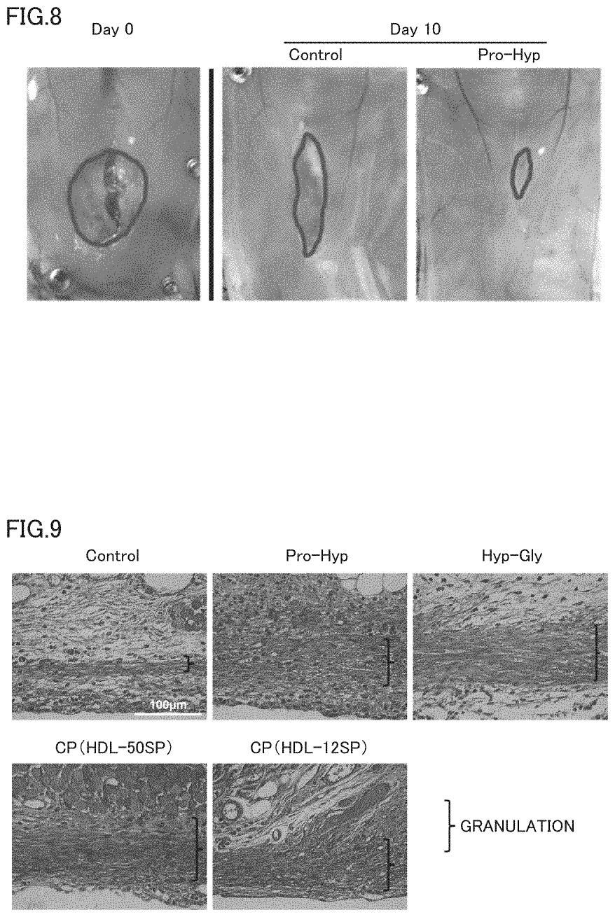

After the formation of the wound site (circular defective injury) in the abdominal wall in Model-2 mice (N=5, per group), a physiological saline solution of Pro-Hyp (500 nmol/200 μL) was administered for 7 days in the same administration schedule as that illustrated in FIG. 4. At this time, physiological saline containing no dipeptide was administered to the control group. 200 μL each of either solution per animal was administered once a day for 7 days, including the day of operation (the day when a wound site was formed, Day 0) (FIG. 4). 200 μL of the physiological saline was administered similarly to the control group (FIG. 4). The wound sites viewed from the abdominal wall side on Day 10 from the day of the wound site formation were compared. The results are shown in FIG. 8. The wound site was reduced with many of them being flat and the healing had been progressing in the Pro-Hyp group in comparison with the control group. The measurement of the area of remaining wound sites revealed that the contraction of the wound sites had significantly progressed in comparison with the control group (Table 5). The areas of wound sites in Table 5 indicate the means determined from 5 mice per group.

TABLE 5

| AREA OF | MEAN | STANDARD |
|---|---|---|
| WOUND SITE | (mm²) | ERROR |
| Day 0 | 31.7 | 3.724 |

| | | | | TEST OF |
|---|---|---|---|---|
| | | | | SIGNIFICANT |
| | | | | DIFFERENCE |
| | | | STAN- | (CONTROL |
| AREA OF | | MEAN | DARD | GROUP VS. |
| WOUND SITE | | (mm²) | ERROR | Pro-Hyp GROUP) |
| Day | CONTROL GROUP | 9.62 | 1.697 | * |
| 10 | Pro-Hyp GROUP | 4.43 | 1.103 | |

* p < 0.05 (student's t-test)

<<Experiment Using Skin Fibroblast-Derived from Murine Fetus>>

<Evaluation of the Degree of Collagen Gel Contraction>

Collagen gel embedded culture of fibroblasts was performed and the degree of collagen gel contraction was measured. The cell line 3T3-L1, which is skin fibroblasts derived from a murine fetus, was cultured using a Dulbecco's modified Eagle medium (D-MEM) (High-Glucose) (manufactured by FUJIFILM Wako Pure Chemical Corporation) containing 10% FBS (fetal bovine serum, manufactured by Bio-Sciences Limited) under conditions at 37° C. and 5% CO₂. A swine-derived type I collagen solution (manufactured by Nitta Gelatin Inc.), a 10 times concentrated D-MEM solution (manufactured by Nitta Gelatin Inc.), and a buffer solution for reconstitution (manufactured by Nitta Gelatin Inc.) were mixed at a volume ratio of 8:1:1 with cooling. Pellets of the above cells recovered using a trypsin solution were seeded therein at a density of 2.5×10⁵ cells/well and solutions prepared at Pro-Hyp concentrations set to the final concentrations were added thereto mixed, 3004 each of which was poured into a 24-well plate. A collagen gel to which neither Pro-Hyp nor cells were added was used as a blank. Subsequently, the plate was incubated for 1 hour at 37° C. under 5% CO₂ condition to gelate collagen. Then, 400 μL of D-MEM (High-Glucose) was gently layered thereover and the collagen gel was separated from the wall surface using a sterilized spatula to have the gel floating in the medium. After further incubation for 69 hours, photographs of the collagen gel were taken with a scanner, and the degree of gel contraction was determined using an image processing software (Image J). The degree of gel contraction was determined by putting a side of the gel in contact with a wall surface of the well and measuring the distance to the opposite wall. The results are shown in Table 6. Since the collagen gel containing fibroblasts contracted by the addition of Pro-Hyp in a concentration-dependent manner, it was revealed that Pro-Hyp has a contracting effect on collagen fibers via fibroblasts. More specifically, it has been suggested that the administration of Pro-Hyp into the peritoneal cavity induces the contraction of collagen fibers via fibroblasts in a wound site expanded by mechanical stress such as tension and results in the promotion of the contraction of the wound site.

TABLE 6

| | Pro-Hyp | MEAN OF CONTRACTION |
|---|---|---|
| | CONCENTRATION | DISTANCE ± |
| FIBROBLASTS | (mM) | STANDARD ERROR (μm) |
| ABSENT | 0 | (98.3 ± 28.5) |
| PRESENT | 0 | (359.7 ± 24.8) |
| | 0.1 | (525.3 ± 56.0) |
| | 1 | (582.1 ± 306.1) |
| | 10 | (1078.9 ± 5.6) |

<Formation of Granulation Tissue by the Administration of Various Polypeptides>

In this experiment, the abdominal wall circular incision model (Model-2) mice were used. Using physiological saline solutions of various polypeptides set forth in Table 7, intraperitoneal administration was performed once a day for 7 days (FIG. 4) and the morphologies of granulation tissue were compared. Moreover, the weight average molecular weights of the polypeptides contained in the collagen hydrolysates were calculated by gel filtration chromatography under the following conditions.

### (Measurement Conditions of Gel Filtration Chromatography)

Mobile phase: 45% acetonitrile (55% water) containing 0.1% trifluoroacetic acid,
Stationary phase: TSK-Gel-2000SWXL column (manufactured by Tosoh Corporation)
Flow rate: 1.0 ml/min,
Column temperature: 40° C.,
Analysis time: 15 minutes,
Injection volume: 10 μl,
Detection wavelength: 214 nm

Thin sections of tissue in a wound site on Day 7 after the formation of the wound site were stained by Masson trichrome stain and granulation tissue was observed. The results are shown in FIG. 9. A possibility was suggested that while the formation of granulation tissue was slight and the distribution of collagen fibers was sparse in the control group (physiological saline), the collagen fibers were dense and matured as the tissue in any of the Pro-Hyp, Hyp-Gly, and collagen hydrolysates administration groups. More specifically, this experiment has suggested that administration of not only a dipeptide such as Pro-Hyp or Hyp-Gly, but also a polypeptide consisting of an amino acid sequence having a dipeptide sequence represented by Pro-Hyp or Hyp-Gly induces the contraction of collagen fibers via fibroblasts in a wound site expanded by mechanical stress such as tension and results in the promotion of the contraction of the wound site.

TABLE 7

| | TEST AGENT | DOSE |
|---|---|---|
| CONTROL | PHYSIOLOGICAL SALINE | 0.2 mL |
| GROUP | | |
| Pro-Hyp | Pro-Hyp (SYNTHETIC | 0.5 μmol/0.2 mL/day ≈ |
| GROUP | PEPTIDE) | 25 μmol/kg/day |
| Hyp-Gly | Hyp-Gly (SYNTHETIC | 0.5 μmol/0.2 mL/day ≈ |
| GROUP | PEPTIDE) | 25 μmol/kg/day |
| CP (HDL- | COLLAGEN HYDROLYSATE | 10 mg/0.2 mL/day ≈ |
| 50SP) | IXOS HDL-50SP | 0.5 g/kg/day |
| GROUP | (MANUFACTURED BY | |
| | Nitta Gelatin Inc.) | |
| | (WEIGHT AVERAGE | |
| | MOLECULAR WEIGHT: | |
| | ABOUT 5600) | |
| CP (HDL- | COLLAGEN HYDROLYSATE | 10 mg/0.2 mL/day ≈ |
| 12SP) | IXOS HDL-12SP | 0.5 g/kg/day |
| GROUP | (MANUFACTURED BY | |
| | Nitta Gelatin Inc.) | |
| | (WEIGHT AVERAGE | |
| | MOLECULAR WEIGHT: | |
| | ABOUT 550) | |

From a series of experimental results described above, it has been suggested that the formation of a hypertrophic scar is inhibited by the following mechanism.

(1) First, administration of a polypeptide (for example, the dipeptide Pro-Hyp) consisting of an amino acid sequence having a dipeptide sequence represented by Pro-Hyp or Hyp-Gly induces the contraction of collagen fibers via fibroblasts in a wound site expanded by mechanical stress (such as tension) and results in the promotion of the contraction of the wound site (Table 6 and FIG. 9).
(2) Since the contracted wound site can be covered with a little granulation tissue, this results in the inhibition of the proliferation of granulation tissue (FIG. 8).
(3) As a result, granulation tissue does not proliferate excessively and the sufficient retraction of granulation tissue occurs at the end of the restoration process to inhibit the formation of hypertrophic scar (FIG. 7).

Conventionally, the proliferation of granulation tissue has been considered to be necessary for wound healing. However, the present inventors have found for the first time that with “granulation tissue of improved quality” that can promote the contraction of the wound site, the wound healing progresses, while the proliferation of the granulation tissue inhibited (that is to say, while the formation of hypertrophic scar inhibited). Furthermore, the present inventors found, for the first time, administering a polypeptide (for example, the dipeptide Pro-Hyp) consisting of an amino acid sequence having a dipeptide sequence represented by Pro-Hyp or Hyp-Gly, in order to improve the quality of granulation tissue and inhibit the formation of hypertrophic scar. The present inventors consider that the excellent effect of inhibiting the formation of the hypertrophic scar by using the polypeptide is unpredictable from the conventional common general technical knowledge and a very different effect from those of such knowledge.

Embodiments and Examples of the present invention are described hereinabove, but it has been planned to combine the configurations of the embodiments and Examples described above as appropriate from the beginning.

The embodiments and Examples disclosed herein are illustrations in all respects and should not be considered to be restrictions. The scope of the present invention is indicated by the claims, but not embodiments and Examples described above, it is intended that all modifications in terms of the meaning of equivalents and the scope of the claims are included.

1. A composition for inhibiting a formation of hypertrophic scar, comprising at least one of a polypeptide consisting of an amino acid sequence having a dipeptide sequence represented by Pro-Hyp or Hyp-Gly, a chemically-modified form thereof, or a pharmaceutically acceptable salt thereof.

2. The composition for inhibiting the formation of hypertrophic scar according to claim 1, wherein the composition is used to inhibit the formation of hypertrophic scar at a site susceptible to mechanical stress.

3. The composition for inhibiting the formation of hypertrophic scar according to claim 2, wherein the site susceptible to mechanical stress comprises at least one selected from the group consisting of abdomen, chest, upper arms, face, and soft tissues.

4. The composition for inhibiting the formation of hypertrophic scar according to claim 1, wherein the polypeptide comprises an oligopeptide consisting of an amino acid sequence of 2 to 20 amino acid residues.

5. The composition for inhibiting the formation of hypertrophic scar according to claim 1, wherein the polypeptide comprises a dipeptide consisting of an amino acid sequence represented by Pro-Hyp or Hyp-Gly.

6. The composition for inhibiting the formation of hypertrophic scar according to claim 1, wherein the polypeptide is a polypeptide derived from natural collagen, a recombinant polypeptide, or a synthetic polypeptide.

7. The composition for inhibiting the formation of hypertrophic scar according to claim 1, wherein the composition is an oral administration formulation, a supplement, a food, or a beverage.

8. The composition for inhibiting the formation of hypertrophic scar according to claim 1, wherein the composition is a transdermal administration formulation, a local administration formulation, an intravenous administration formulation, or a cosmetic preparation.

9. A method for inhibiting a formation of hypertrophic scar, comprising administering an effective amount of at least one of a polypeptide consisting of an amino acid sequence having a dipeptide sequence represented by Pro-Hyp or Hyp-Gly, a chemically-modified form thereof, or a pharmaceutically acceptable salt thereof to a subject in need thereof.

10. The method for inhibiting the formation of hypertrophic scar according to claim 9, wherein the method is used to inhibit the formation of hypertrophic scar at a site susceptible to mechanical stress.

11. The method for inhibiting the formation of hypertrophic scar according to claim 10, wherein the site susceptible to mechanical stress comprises at least one selected from the group consisting of abdomen, chest, upper arms, face, and soft tissues.

12. The method for inhibiting the formation of hypertrophic scar according to claim 9, wherein the polypeptide comprises an oligopeptide consisting of an amino acid sequence of 2 to 20 amino acid residues.

13. The method for inhibiting the formation of hypertrophic scar according to claim 9, wherein the polypeptide comprises a dipeptide consisting of an amino acid sequence represented by Pro-Hyp or Hyp-Gly.

14. The method for inhibiting the formation of hypertrophic scar according to claim 9, wherein the polypeptide is a polypeptide derived from natural collagen, a recombinant polypeptide, or a synthetic polypeptide.
